(19) United States
(12) Patent Application Publication
(10) Pub. No.: US 2005/0143259 A1
(43) Pub. Date: Jun. 30, 2005

(54) Thermal weed, fungal, insecticidal and sterilisation method

(76) Inventor: Richard John Newson, Hastings (NZ)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/747,868

(22) Filed: Dec. 29, 2003

(51) Int. Cl. A01N 25/00; A61L 2/08

(52) U.S. Cl. 504/116.1; 422/27

(57) ABSTRACT

A method for killing or controlling weeds, insects, fungi, the release of spores, bacteria or sterilising a substrate is disclosed. The method includes the step of exposing the weeds, fungi, insects or insect eggs, spores or substrate to heat. A second step performed immediately after, before, or simultaneously with the heating step includes applying a liquid substance selected from a herbicide and/or pesticide and/or fungicide, and/or bacteriacide, and/or fish or animal-derived substance to the weeds, fungi, insects, spores or substrate.

### FIELD OF INVENTION

The invention comprises a method for killing or controlling weeds, fungi, the release of spores, bacteria, or sterilizing a substrate, particularly in agricultural including horticultural and viticultural applications, but also other industrial, commercial, and domestic applications.

### BACKGROUND

Weeds may be controlled by pulling out the weeds or by use of chemical sprays. There is a growing awareness however of side effects which may arise from chemical sprays. International patent application publication WO 96/03036 discloses a weed killing method which comprises blowing hot air containing a small amount of water onto the weeds.

Chemical sprays or treatments are also used against fungi and bacteria for example, in agricultural, horticultural, viticultural, commercial, industrial, domestic and similar applications. For example, in horticulture lime sulphur sprays are used to combat the fungal disease Venturia inaequalis also known as blackspot or apple scab which infects the foliage and fruit of apple trees. Leaves infected with this organism falling to the ground in autumn release ascospores in spring after raining, which can reinfect the new spring foliage and fruit on the trees.

### SUMMARY OF INVENTION

The invention provides an improved or at least alternative method for killing or controlling weeds and/or fungi and/or the release of spores, which is effective to control Venturia inaequalis in orchard apple trees in particular, and may also be useful for killing insect pests and for sterilizing substrates generally, which is likely to find application in agriculture including horticulture and viticulture at least.

In broad terms in one aspect the invention comprises a method for killing or controlling weeds, insects, fungi, the release of spores, bacteria or sterilising a substrate, including exposing the weeds, fungi, insects or eggs thereof, spores or substrate to heat and immediately thereafter or before or simultaneously applying a liquid substance including a herbicide and/or pesticide and/or fungicide, and/or bacteriacide, and/or fish or animal-derived substance to the weeds, fungi, insects, spores or substrate.

In broad terms in another aspect the invention comprises a method for killing or controlling weeds including heating the weeds and immediately thereafter or before on simultaneously applying a liquid herbicide and/or an animal or fish-derived substance onto the weeds.

In broad terms in another aspect the invention comprises a method for killing or controlling weeds including heating the weeds and immediately thereafter or before or simultaneously applying an oil onto the weeds.

In broad terms in a further aspect the invention comprises a method for killing or controlling fungi or the release of spores, including blowing onto a substrate on which the fungi or spores exist hot air, hot air containing water, water vapour or steam, and immediately thereafter or before or simultaneously applying a liquid substance including a fungicide and/or an animal or fish-derived substance onto the substrate.

In broad terms in a further aspect the invention comprises a method for killing or controlling fungi or the release of spores including heating a substrate on which the fungi or spores exist and immediately thereafter or before or simultaneously applying an oil onto the substrate.

Preferably the method includes exposing the weeds, fungi, insects or eggs, spores or substrate to heat by blowing hot air or hot air containing water, water vapour, or steam onto the weeds, fungi, insects or eggs, spores, or substrate. Alternatively exposing the weeds, fungi, insects or eggs, spores or substrate to heat may include exposing the weeds, fungi, insects or eggs, spores, or substrate to steam or to a radiant heat source.

Preferably the method includes exposing the weeds, fungi, insects or eggs, spores or substrate to heat above 100° C. and most preferably above 450° C.

Preferably the liquid substance includes one or more of a herbicide, a fungicide, a bactericide, and an oil such as a fish oil.

The method may include applying the liquid substance to ground in which weeds are growing or to a substrate on which fungi or spores, or insects or eggs exist, at a rate of between 5 and 50 litres per hectare of surface area, or at a rate of between 10 and 30 litres per hectare of surface area.

Preferably the liquid substance wets weeds, fungal material, or a substrate sufficiently to prevent ignition thereof.

Preferably the method includes heating the weeds, fungi, spores or substrate and immediately thereafter applying the liquid substance.

### BRIEF DESCRIPTION OF THE FIGURES

The invention is further described with reference to the accompanying drawings by way of example and without intending to be limiting, wherein:

FIG. 1 illustrates carrying out the method of the invention against weeds,

FIG. 2 is a cross-sectional view through one form of apparatus for use in the method of the invention,

FIG. 3 is a close up cross-sectional view of another form of apparatus for use in the method of the invention,

FIG. 4 illustrates carrying out the method of the invention in a vineyard to prevent release of spores of Botrytis from leaf litter on the ground between rows of vines, using a tractor mounted apparatus, and

FIG. 5 shows one form of tractor mounted apparatus from one side.

### DETAILED DESCRIPTION OF PREFERRED FORMS

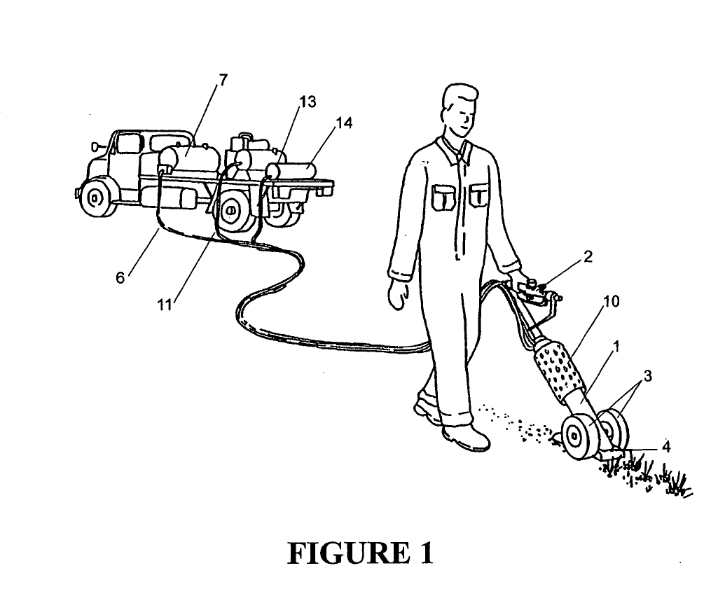

FIGS. 1-3 illustrate one form of apparatus suitable for carrying out the method of the invention particularly against weeds. Referring to the drawings, the apparatus comprises a metal cylinder 1, the interior of which forms a hollow chamber in which the air is heated by a gas burner. At one end the apparatus also comprises a handle 2 and optionally at the other end ground wheels 3. The outlet 4 may optionally be surrounded by a shroud which extends rearwardly as shown.

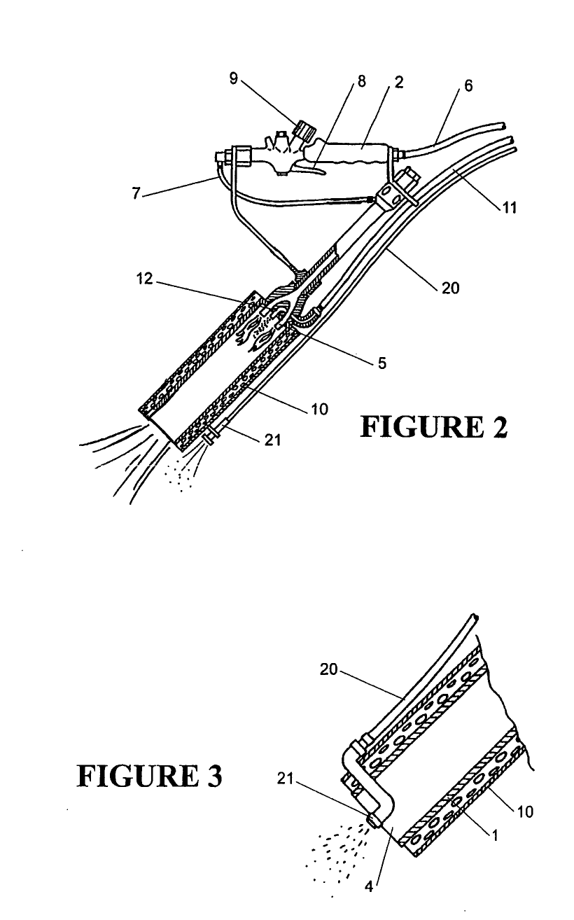

Gas burner outlets 5 are provided at the end of the chamber 1 opposite the open end 4. In use of the apparatus the gas burner outlets 5 create a flame at the upper end of the chamber as shown in FIG. 2. The gas burner outlets 5 are supplied with gas such as LPG or propane or similar over a gas supply line 6 from for example a truck mounted gas tank 7 as shown in FIG. 1. From the apparatus shown the gas supply line 6 connects to the handle 2 and the gas passes through the interior of the handle and through conduit 7 to the gas burners 5.

The handle 2 also incorporates a trigger switch 8 which controls the gas, air and flow of the liquid substance, and optionally a gas adjustor 9 and/or an adjustor (not shown) for the flow rate of the liquid substance. An operator can turn the gas, air, and liquid substance flow on or off using the trigger switch 8 as the operator rolls the apparatus over weeds, or matter infected with fungi or carrying spores such as leaf litter on the ground in an orchard or vineyard or a surface otherwise to be sterilised to kill bacteria or insects vermin for example. When the trigger 8 is released so that the gas and air flow is “off”, either a small amount of gas is still allowed to flow to the gas burners 5 to maintain effectively a pilot flame, so that when the trigger is again pulled the gas will immediately ignite or alternatively the trigger may also activate a piezo electric spark starter to relight the gas flow. The primary air source for combustion at the gas burners is ambient air which can pass through a heat shield 10 around the top end of the cylinder 1, and into the chamber through holes formed in the top part of the cylinder. A compressed air line 11 supplies a flow of compressed air to a compressed air inlet 12 at the top of the chamber 1 as shown. The compressed air flow is also controlled by the trigger 8 but with a slight delay, of for example five seconds or so, so that when the trigger 8 is pulled to “light” the burners 5 to a maximum, after a short delay to allow the burners to light, compressed air will also flow from the inlet 12. The air will flow through the flames from the burners 5 so that the high pressure air entering the top end of the chamber from the compressed air inlet 12 and flowing through the chamber 1 is heated, to for example of the order of 450 to 750° C.

A supply line 20 supplies a flow of the liquid substance to an outlet nozzle or nozzles 21 at the bottom of the chamber as shown behind the hot air outlet. Compressed air is supplied from a compressor 13 mounted on the back of the truck as shown. The liquid substance is supplied from a tank 14. Instead of air being supplied from a compressor over an air hose to the compressed air outlet to cause the air flow through the chamber, in an alternative form a large fan could be mounted on the truck or a four wheel ATV which supplies air over a large diameter pipe to the apparatus such as a pipe of approximately 100 mm diameter for example. In another form a high temperature electric heating element instead of a gas burner maybe provided in the top end of the interior of the cylinder 1 or equivalent.

As the apparatus passes over the weeds or infected leaf litter example, the weeds or leaf litter are exposed to a blast of hot air. Immediately thereafter as the apparatus passes over the weeds or leaf litter the liquid substance is sprayed onto the weeds or leaf litter from the nozzle or nozzles 21 or equivalent. Where it is intended to kill or control weeds, the liquid substance will be a herbicide and/or an animal or fish-derived substance such as an animal or fish-derived oil, or might alternatively be a mineral oil. A herbicide including a terpene component including in particular a monoterpene may be employed, and/or a fatty acid-based component and in particular a fatty acid soap. Optionally the herbicide may include a foaming agent. A particularly preferred liquid substance for use in the method of the invention against weeds is a herbicide based on a terpene component, a fatty acid soap, and optionally a foaming agent as particularly disclosed in international patent application publication WO 99/53764, the entire content of which is incorporated herein by reference, and sold as ORGANIC INTERCEPTOR™ by Certified Organics Ltd, New Zealand. Other organic or non-organic herbicides may be used however, as well as other substances with herbicidal activity which may occur naturally as liquids or may be dissolved in water or another solvent or mixed as a suspension to enable them to be sprayed onto the weeds or substrate as described.

An advantage of using a fish or animal oil is that in a horticultural or viticultural application for example the decaying plant matter coated with the oil will add nutrients to the soil which is enhanced by the use of an animal or fish oil.

FIG. 3 shows apparatus similar to that of FIG. 2 but where in FIG. 3 the outlet nozzle 21 is positioned within the hot air outlet of the chamber, to inject the liquid substance into the hot air flow, so that the liquid substance is mixed as a spray or hot mist or vapour with the hot air flow.

Without intending to be limiting, it is believed that exposure to heat such as the blast of hot air breaks down the outer membrane or cellular structure of weeds so that the liquid substance which is sprayed onto the weeds immediately thereafter can more effectively penetrate into or coat the weeds. A more effective kill is achieved and/or where the liquid substance is a herbicide, fungicide, pesticide, bactericide or similar a substantially lower volume of the herbicide, fungicide, pesticide, or bactericide can be used, giving important economic benefits and in some cases environmental benefits through lower chemical use.

FIG. 1 shows carrying out of the method of the invention against weeds, using apparatus as described. In alternative forms the wheels 3 may be eliminated and the apparatus maybe carried by the operator and directed over the weeds or ground or substrate to be sterilized. The apparatus shown in FIGS. 1 to 3 by way of example is towards the larger end of the scale for handheld apparatus and alternatively the method of the invention maybe carried out particularly against the weeds using smaller gun-size apparatus intended to be hand-held, or alternatively again by way of a larger vehicle mounted or towed apparatus. For example a series of hot air outlets or a laterally extending hot air outlet and one or more spray nozzles to cover the same area may be mounted, typically beneath a shroud or shrouds, across the front or rear of a tractor or four wheel all terrain vehicle (ATV) for use in larger scale applications. For example a tractor or ATV mounted apparatus may be used for sterilizing leaf litter between rows of trees in an orchard or vines in a vineyard. Larger tractor mounted apparatus maybe used for killing weeds over larger areas of ground prior to cultivation. Truck mounted apparatus maybe used for killing weeds along a pavement edge or verge of a road. The method of the invention maybe used in various such applications.

While in the embodiment described above the trigger 8 controls both the flow of hot air of the liquid substance, in an alternative form a trigger or other manual control may enable the operator to selectively apply the liquid substance, so that the liquid substance is sprayed intermittently while the hot air flow is operating. This may enable an operator killing weeds growing through a pavement or pathway or other paved area to leave the hot air flow running but selectively apply the liquid substance as the apparatus passes over individual weeds growing through the paved area.

FIG. 2 shows the nozzle 21 for spraying the liquid substance positioned a few centimetres behind the hot air outlet 4, but to increase the time delay between exposure of the weeds, fungi, spores or substrate to the heat and then to the spray of the liquid substance the nozzle or nozzles 21 or equivalent may be spaced further behind the hot air outlet via a larger or longer rearwardly extending mounting bracket (so that the time delay is greater). It is believed to be preferable for the weeds or substrate to be hot or warm from the hot air at the time that the liquid substance is applied. Another advantage of applying the liquid substance immediately after exposure of the weeds or substrate to the hot air is that liquid substance can wet the weeds, fungi, or substrate sufficiently to prevent ignition (flaming) of the weeds, leaf litter, or similar. In a yet alternative embodiment in some applications it maybe desirable to apply the liquid substance to the weeds, fungi, spores or substrate immediately before exposure to the hot air. The liquid substance will usually be applied as a light spray or mist or a heavier shower where the substance is more heavily diluted with water for example, and may be preheated so that it is applied as a hot mist or vapour.

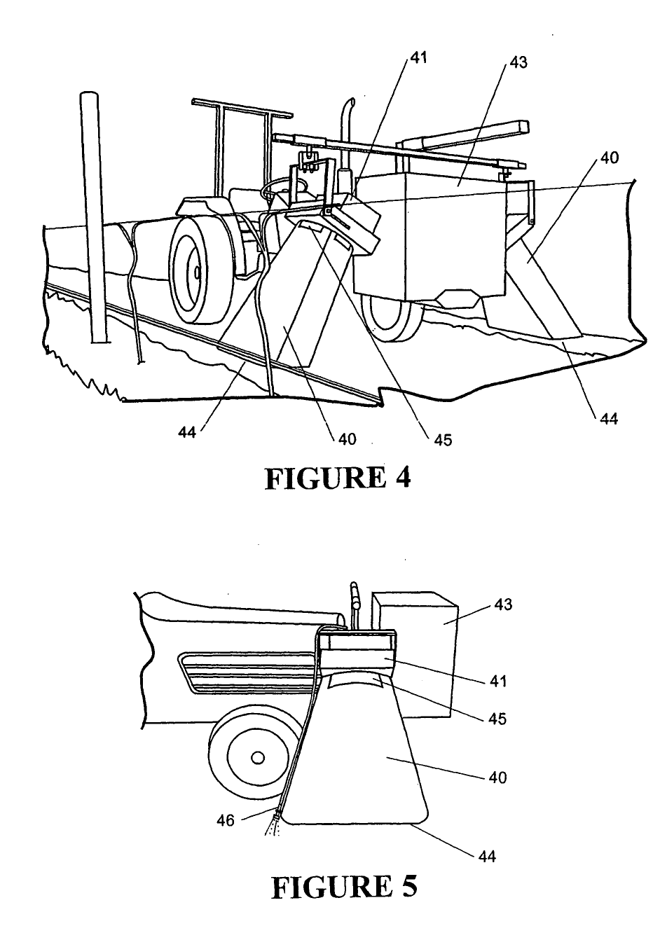

FIGS. 4 and 5 show another form of apparatus suitable for carrying out the method of the invention, which is vehicle mounted, FIG. 4 illustrating carrying out the method of the invention in a vineyard to prevent weed growth beneath vines and release of spores of Botrytis from leaf litter on the ground between the rows of vines. Mounted on either side of the vehicle which in this case is a tractor are large stainless steel chambers 40 having a diesel burner and a blower mounted at the upper end thereof beneath cover 41. The diesel burner burns diesel fuel supplied from a tank 43 creating a flame in the top of the interior of the chamber 40 which heats air from the blower, which is blown from the outlet 44 of the apparatus onto the ground along rows of vines on either side of the vehicle as the vehicle moves between the rows. Water is also injected into the top of the chambers 40 so that the air exiting the lower ends of the chambers contains more moisture, water, water vapour, or steam. The water may be injected as a spray or mist into the tops of the chambers 40 or lower down within the chambers as described in international patent application publication WO 96/03036 the entire content of which is incorporated herein by reference or alternatively the water may pass through a metal coil positioned within the top of each chamber 40 and which is heated by the diesel flame so that the water is heated to boiling and above within the metal coil and exits the metal coil into the airflow at the top of the chamber as superheated steam. The tank 43 has two chambers, one for fuel to feed the burner and the other to provide a water supply. Preferably apertures or air gaps 45 through the top of the chambers 40 enable the air in the top of the chambers which is heated and expanding passing down the chambers as an air blast to draw in more air via a venturi effect. Nozzles 46 (see FIG. 5) for spraying the liquid substance is positioned behind each chamber 40, which spray the liquid substance as described previously onto the weeds or leaf litter immediately after exposure to the hot air containing water vapour.

Instead of exposing the weeds or leaf litter or other substrate to a hot air blast or a hot air blast containing water or water vapour or steam as described, alternatively the weeds or leaf litter or substrate may be heated by being exposed to steam including superheated steam, or to radiant heating from a hot plate which is passed over the weeds or leaf litter or substrate. A hot plate may be heated by electric elements incorporated within the hot plate, or by gas or diesel burners which project a flame or flames towards the hot plate from above, preferably within a shroud around the hot plate. In another variant to create steam or hot air containing water vapour, water may be sprayed onto the hot plate so that the water will evaporate within such a shroud, so that as the apparatus passes over for example weeds, the weeds are exposed to hot moist air or steam.

The method of the invention has been found to be effective to control Venturia inaequalis also known as black spot, in apple orchards. Leaf litter on the ground in an orchard, or remaining leaf litter after the bulk of fallen leaves have been removed, is exposed to heat and a fungicide or an animal or fish-derived substance and preferably an oil such as a fish oil, which has been found effective to prevent the release of ascospores from the leaf litter during the subsequent spring. One fish-derived substance which has been found to be particularly effective to suppress black spot ascospore discharge from apple leaf litter in orchards is BIO-SEA™ sold by Sealord Group Limited, New Zealand, which is a liquid substance including fish oil.

Indications are that the same treatment is effective to combat by Botlrytis cinerea in vineyards, by again treating the ground and any leaf litter thereon immediately below vines in the same way.

Also indications are that the method of the invention is effective to combat grape mealybug or Pseudococcus maritimus which affects grape vines and also orchard fruit and trees. The bugs and/or eggs in New Zealand commonly reside in clover which grows along the grape vines or beneath orchard trees, and can be reduced or eradicated by heat treatment followed by spraying with a liquid substance such an oil or alternatively a pesticide or substance having some degree of pesticide or properties.

The following description of trials work further illustrates the invention:

Use Against Fungal Disease and Spore Release

Leaf litter was collected into wire mesh sandwiches and monitored for ascospore discharge. The leaf litter was collected from beneath Royal Gala trees at Te Moe Orchard, Hawkes Bay, New Zealand and sandwiched between twelve, wire-mesh squares, each approximately 240 by 160 mm. The trial orchard had been severely affected by black spot the previous season. The litter squares were laid on the ground and treated by method of the invention using the apparatus previously described. A control group of litter squares received no treatment. A second group of litter squares was treated according to the method of the invention using apparatus as previously described, by application of air heated by a diesel burner, generally as described with reference to FIGS. 4 and 5, immediately followed by spraying with BIO-SEA™ fish oil product available from Sealord Group Limited, New Zealand, at the rate of 20 litres/hectare. A third group of litter squares was treated with air heated by a gas burner only, without application of oil, and a fourth group of litter squares was treated by spraying BIO-SEA™ fish oil onto the litter squares only, without hot air application.

After treatment the litter squares were laid out on a mown lawn with three numbered microscope slides randomly placed over each. After each of a number of subsequent daytime rainfall events, the slides were removed, replaced by fresh slides, and scanned microscopically for ascospores of Venturia inaequalis. These are readily differentiated from impacted spores of other species by their characteristic shape. Each treatment was thus assessed from nine slides (replicates), counted and replaced five times from over approximately a three week period. The mean number of ascospores impacted per square cm per slide were analysed by ANOVA, and LSD values (5% probability), calculated for each.

The results are shown in Table 1 below. Mean discharge of untreated litter squares over the five rainfalls was 174 per cm². Hot air treatment reduced this to 32 per cm², and fish oil alone also, to 92 per cm². The combination treatment completely suppressed discharge.

TABLE 1
Ascospores per square cm

| Treatment | Mean | 13-Oct | 17-Oct | 25-Oct | 30-Oct | 2-Nov |
|---|---|---|---|---|---|---|
| Untreated | 174.0 | 162.2 | 315.0 | 29.1 | 119.7 | 244.0 |
| Hot Air | 31.9 | 60.9 | 69.4 | 2.6 | 0.3 | 26.0 |
| BIO-SEA ™ | 92.0 | 54.8 | 150.9 | 68.6 | 58.2 | 128.0 |
| 20L/ha Hot Air | 0.0 | 0.2 | 0.0 | 0.0 | 0.0 | 0.0 |
| and BIO-SEA ™ | | | | | | |
| LSD | 51.7 | 100.6 | 167.2 | 52.1 | 54.9 | 91.8 |

For the combination treatment, apart from the first rainfall when three ascospores (one on each of three slides) were counted, the results for successive rainfalls were consistent, with no ascospores being detected.

Use Against Weeds

Common pasture weeds in Hawkes Bay, New Zealand were treated as follows. A first strip of weeds was treated with hot air only, heated by a diesel burner. A second adjacent strip of weeds was sprayed with ORGANIC INTERCEPTOR™ herbicide product available from Certified Organics Limited, New Zealand, diluted in water to about 20% strength, and using a conventional hand held weed sprayer. A third adjacent strip of weeds was treated with hot air heated by a diesel burner and was immediately thereafter sprayed with ORGANIC INTERCEPTOR™ diluted in water to about 5% strength.

Weather conditions at the time of treatment were dry and fine and remained so for four days after treatment. The effect of the treatments on the weed strips was monitored for ninety days after treatment.

The results are shown in Table 2 below, in which the figures given indicate the approximate height of the weeds before and at each assessment time after treatment. That is, before treatment most weeds in the weed strips which were treated had heights in the range 80-100 mm. Hot air treatment alone killed the weeds/reduced the weed height to under 10 mm after 7-14 days, following which regrowth began to occur. Spraying with ORGANIC INTERCEPTOR™ diluted in water to 20% strength killed the weeds/reduced weed height to under 15 mm in 14-30 days after which regrowth began to occur. Hot air treatment combined with spraying of ORGANIC INTERCEPTOR™ diluted in water to 5% strength killed all weeds completely with minimal regrowth up to 60-90 days. As well as killing the weeds it also substantially rapidly reduced the height and bulk of the dead weeds (within hours as opposed to weeks).

TABLE 2

| | | | 24 hours | 7 days | 14 days | 30 days | 44 days | 60 days | 90 days |
|---|---|---|---|---|---|---|---|---|---|
| | Before | After | after | after | after | after | after | after | after |
| | Treatment | Treatment | treatment | treatment | treatment | treatment | treatment | treatment | treatment |
| Hot Air | 80-100 | 20-40 | 10-30 | 0-10 | 0-10 | 5-10 | 10-20 | 20-40 | 50-00 |
| ORGANIC | 80-100 | 80-100 | 10-30 | 80 | 0-15 | 0-15 | 5-20 | 10-30 | 30-50 |
| INTERCEPTOR ™ | | | | | | | | | |
| diluted in water to | | | | | | | | | |
| 20% strength | | | | | | | | | |
| Hot Air and | 80-100 | 20-30 | 10-30 | 0-15 | 0-0 | 0-0 | 0-0 | 0-5 | 0-5 |
| ORGANIC | | | | | | | | | |
| INTERCEPTOR ™ | | | | | | | | | |
| diluted in water to | | | | | | | | | |
| 5% strength | | | | | | | | | |

The foregoing describes the invention including a preferred form thereof. Alterations and modifications as will be obvious to those skilled in the art are intended to be incorporated within the scope hereof.

1. A method for killing or controlling weeds, insects, fungi, the release of spores, bacteria or sterilising a substrate, including exposing the weeds, fungi, insects or insect eggs, spores or substrate to heat and immediately thereafter or before or simultaneously applying a liquid substance including a herbicide and/or pesticide and/or fungicide, and/or bacteriacide, and/or fish or animal-derived substance to the weeds, fungi, insects, spores or substrate.

2. A method according to claim 1 including exposing the weeds, fungi, insects or eggs, spores or substrate to heat by blowing hot air onto the weeds, fungi, insects, spores, or substrate.

3. A method according to claim 1 including exposing the weeds, fungi, insects or eggs, spores or substrate to heat by blowing hot air containing water, water vapour, or steam onto the weeds, fungi, insects, spores, or substrate.

4. A method according to claim 1 including exposing the weeds, fungi, insects or eggs, spores or substrate to heat by exposing the weeds, fungi, insects, spores, or substrate to steam.

5. A method according to claim 1 including exposing the weeds, fungi, insects or eggs, spores or substrate to a radiant heat source.

6. A method according to claim 1 including exposing the weeds, fungi, insects or eggs, spores or substrate to heat above 100° C.

7. A method according to claim 1 including exposing the weeds, fungi, insects or eggs, spores or substrate to heat above 450° C.

8. A method according to claim 1 wherein the liquid substance includes a herbicide.

9. A method according to claim 8 wherein the herbicide includes a terpene-based component.

10. A method according to claim 9 wherein the liquid substance includes a fatty acid-based component.

11. A method according to claim 10 wherein the fatty acid-based component is a fatty acid soap.

12. A method according to claim 11 wherein the liquid substance also includes a foaming agent.

13. A method according to claim 1 wherein the liquid substance includes a fungicide.

14. A method according to claim 1 wherein the liquid substance includes an oil.

15. A method according to claim 14 wherein the liquid substance includes a fish oil.

16. A method according to claim 1 including applying the liquid substance to ground in which weeds are growing or to a substrate on which fungi or spores or insects or insect eggs exist at a rate of between 5 and 50 litres per hectare of surface area.

17. A method according to claim 1 including applying the liquid substance to ground in which weeds are growing or to a substrate on which fungi or spores or insects or insect eggs exist at a rate of between 10 and 30 litres per hectare of surface area.

18. A method according to claim 1 wherein the liquid substance wets weeds, fungal material, or a substrate sufficiently to prevent ignition thereof.

19. A method according to claim 1 including heating the weeds, fungi, spores or substrate and immediately thereafter applying the liquid substance.

20. A method according to claim 1 including heating the weeds, fungi, spores or substrate while simultaneously exposing same to a spray, mist, or vapour of the liquid substance.

21. A method according to claim 1 including controlling the application of the liquid substance separately from control of application of the heat, via a manual user-operable control.

22. A method according to claim 1 including applying the heat and liquid substance from a hand held appliance carried by a user.

23. A method according to claim 1 including applying the heat and liquid substance from an applicator carried or towed by a motorised vehicle.

24. A method for killing or controlling weeds including heating the weeds and immediately thereafter or before on simultaneously applying a liquid herbicide and/or an animal or fish-derived substance onto the weeds.

25. A method according to claim 24 including exposing the weeds to heat by blowing hot air onto the weeds.

26. A method according to claim 24 including exposing the weeds to heat by blowing hot air containing water, water vapour or steam onto the weeds.

27. A method according to claim 24 including exposing the weeds to heat by exposing the weeds to steam.

28. A method according to claim 24 including exposing the weeds to a radiant heat source.

29. A method according to claim 24 including exposing the weeds, fungi, insects, spores or substrate to heat above 100° C.

30. A method according to claim 24 including exposing the weeds, fungi, insects, spores or substrate to heat above 450° C.

31. A method according to claim 24 wherein the herbicide includes a terpene-based component.

32. A method according to claim 24 wherein the herbicide includes a fatty acid-based component.

33. A method according to claim 32 wherein the fatty acid-based component is a fatty acid soap.

34. A method according to claim 33 wherein the liquid substance also includes a foaming agent.

35. A method for killing or controlling weeds including heating the weeds and immediately thereafter or before or simultaneously applying an oil onto the weeds.

36. A method according to claim 35 wherein the oil is a fish oil.

37. A method for killing or controlling fungi or the release of spores, including blowing onto a substrate on which the fungi or spores exist hot air, hot air containing water, water vapour or steam, and immediately thereafter or before or simultaneously applying a liquid substance including a fungicide and/or an animal or fish-derived substance onto the substrate.

38. A method according to claim 37 wherein the substrate is leaf litter on the ground in an orchard on which exists spores of Ventura inaequalis.

39. A method according to claim 37 wherein the substrate is leaf litter on the ground in an vineyard on which exists spores of Botrytis cinerea.

40. A method for killing or controlling insect pests or the eggs thereof including heating a substrate on which the insects or eggs thereof exist and immediately thereafter or before or simultaneously applying a herbicide, pesticide, or an oil to the substrate.

41. A method according to claim 40 wherein the insect pest is grape mealybug (Pseudococcus maritimus).
